(19) United States
(12) Patent Application Publication
(10) Pub. No.: US 2026/0120037 A1
(43) Pub. Date: Apr. 30, 2026

(54) Advanced Forecasting Tool for Key Performance Indicators in Revenue Cycle Management

(71) Applicant: Cerner Innovation, Inc., Kansas City, MO (US)

(72) Inventors: Suman Pal, Bangalore (IN); Rupanjali Chaudhuri, Bangalore (IN); Chetan KV, Bangalore (IN); Monica Gaur, Delhi (IN)

(73) Assignee: Cerner Innovation, Inc., Kansas City, MO (US)

(21) Appl. No.: 19/093,670

(22) Filed: Mar. 28, 2025

Related U.S. Application Data
(60) Provisional application No. 63/712,909, filed on Oct. 28, 2024.

(51) Int. Cl. G06Q 10/0639; G06Q 10/04; G16H 10/60

(52) U.S. Cl. CPC G06Q 10/06393; G06Q 10/04; G16H 10/60

(57) ABSTRACT

Techniques for generating datasets for training models for forecasting RCM KPIs are disclosed. Initially, the system accesses a set of healthcare data associated with one or more KPIs. A first KPI is represented by time series data points. The system generates a plurality of datasets by applying a sliding window of order “N” to the time series data points. The system determines IQR scores for datasets of a set of “N” datasets that include a first data point. The system determines threshold ranges for the datasets of the set of “N” datasets. Responsive to the first data point being outside the threshold ranges for the datasets, the system selects the first data point as a first outlier. The system replaces the outlier in the plurality of time series data points to generate an aggregated dataset that is used to train a machine learning model to forecast the first KPI.

### BENEFIT CLAIMS; RELATED APPLICATIONS; INCORPORATION BY REFERENCE

This application claims the benefit of U.S. Provisional Patent Application 63/712,909, filed Oct. 28, 2024, which is hereby incorporated by reference.

The Applicant hereby rescinds any disclaimer of claim scope in the parent application(s) or the prosecution history thereof and advises the USPTO that the claims in this application may be broader than any claim in the parent application(s).

### TECHNICAL FIELD

The present disclosure relates to artificial-intelligence-driven healthcare management systems and processes. In particular, the present disclosure relates to handling outliers and accounting for external factors in healthcare data when training machine learning models.

### BACKGROUND

Revenue Cycle Management (RCM) in U.S. healthcare refers to the process of managing the transactional aspects of healthcare services provided to patients, from the initial appointment scheduling and registration to the final payment collection. RCM involves various steps such as patient registration, insurance verification, coding and billing, claims processing, payment collection, and accounts receivable management.

Maintaining good operational efficiency of healthcare organizations requires forecasting of key performance indicators (KPIs) such as revenue, cash flow, and footfall. Revenue in healthcare refers to the total income generated from providing medical services to patients. Revenue includes payments received from patients, insurance companies, government healthcare programs, e.g., Medicare and Medicaid, and other sources. Cash flow in healthcare refers to the movement of money in and out of a healthcare organization over a specific period. Cash flow includes cash receipts from patient payments, insurance reimbursements, investments, and other sources, as well as cash disbursements for operating expenses, equipment purchases, debt servicing, and other obligations. Footfall, also known as patient volume or visitation, refers to the number of patients or visitors entering a healthcare facility within a given period.

Software applications may automate and streamline various aspects of RCM operations. For example, software applications may include robotic process automation (RPA) technology to automate rule-based tasks, such as eligibility verification and patient registration. Artificial intelligence (AI) and machine learning (ML) may also be used to analyze data, learn patterns, and formulate predictions to help optimize workflows.

Building robust AI and ML models into such systems, however, is complicated due to the difficulty in accessing accurate and complete healthcare data. Outliers are data points that deviate significantly from an expected trend or distribution, which can negatively impact the training of ML models. For instance, outliers in healthcare data may dominate or otherwise skew a learning process, leading the ML model to overfit the points at the expense of model performance. Adding to the technical complexity, not every outlier is an error in healthcare. Some outliers may represent anomalies while others may represent rare clinical cases. External factors, such as seasonal trends, economic changes, and regulatory updates, may also negatively affect ML model training and performance if the ML algorithms are not robust enough to handle such changes in the input data.

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1A illustrates a system in accordance with one or more embodiments;

FIG. 1B illustrates a machine learning engine in accordance with one or more embodiments;

FIG. 2 illustrates an example set of operations of the machine learning engine of FIG. 1B;

FIG. 3 illustrates an example set of operations for generating datasets for training forecasting models in accordance with one or more embodiments;

FIG. 4A illustrates details for calculating an IQR score for a sample dataset,

FIG. 4B illustrates details for calculating IQR threshold ranges for the sample dataset;

FIGS. 5A and 5B show a selection of engineered features;

FIG. 6 identifies various forecasting models and pros and cons for the models; and

FIG. 7 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. KEY PERFORMANCE INDICATOR FORECASTING SYSTEM ARCHITECTURE
3. MACHINE LEARNING ARCHITECTURE
4. MACHINE LEARNING ENGINE OPERATION
5. GENERATIVE MODELS
6. GENERATING DATASETS FOR TRAINING FORECASTING MODELS
7. EXAMPLE IQR CALCULATIONS
8. EXAMPLE ENGINEERED FEATURES
9. VARIOUS FORECASTING MODELS
10. PRACTICAL APPLICATION; IMPROVEMENTS & ADVANTAGES
11. HARDWARE OVERVIEW
12. MISCELLANEOUS; EXTENSIONS

### 1. General Overview

One or more embodiments generate a dataset for training an ensemble of models for forecasting revenue cycle management (RCM) key performance indicators (KPIs) in healthcare. RCM refers to the process of managing transactional aspects of healthcare services provided to patients. KPIs, as referred to herein, include revenue, cash flow, footfall, claim denial rates, claim turnaround times, and/or other metrics relating to operational efficiency. The training process identifies outliers in RCM data and applies techniques for replacing the outliers. The training process also implements techniques for addressing external factors. The techniques provide a robust ML model that can handle changes in the input RCM data, such as noisy data, missing values, or shifts in data distribution, without significant performance degradation. By optimizing the model's ability to maintain stable and reliable performance despite challenges arising in the healthcare data provided as input, the system may deliver improved AI-driven guidance and/or automation directed at improving operational efficiency and patient service delivery.

Initially, the system accesses a set of healthcare data associated with one or more KPIs. A first KPI is represented by time series data points. The system generates a plurality of datasets by applying a sliding window of order “N” to the time series data points. The system identifies outliers by determining a set of “N” datasets of the plurality of datasets that include a first data point. The system determines interquartile range (IQR) scores for the datasets of the set of “N” datasets. Using the IQR scores for the respective datasets, the system determines threshold ranges for the datasets of the set of “N” datasets. Responsive to the first data point being outside the threshold ranges for the datasets of the set of “N” datasets, the system selects the first data point as a first outlier. The system replaces the outlier in the plurality of time series data points to generate an aggregated dataset for the first KPI. The aggregated dataset is used to train at least one machine learning model to forecast the first KPI.

One or more embodiments determine a replacement value for replacing the first outlier in the plurality of time series data points. The system identifies the neighboring data points on either side of the first outlier. A median of the neighboring data points is calculated and is used as a replacement value for the first outlier. The system excludes outliers from the neighboring data points.

One or more embodiments determine threshold ranges for datasets of set of “N” datasets by first arranging the data points in the dataset in ascending order. A Q1 value, i.e., 25^{th} percentile, and a Q3 value, i.e., 75^{th} percentile, is determined for each of the datasets. The system subtracts the Q3 value from the Q1 value to determine an IQR score. A lower threshold of the threshold range is calculated by subtracting, 1.5 times the IQR score from the Q1 value and an upper threshold of the threshold range is calculated by adding 1.5 times the IQR score to the Q3 value.

One or more embodiments train an ensemble of ML forecasting models. An ML forecasting model, also referred to herein as a predictive model, refers to a computer program or object that has been trained, via one or more machine learning algorithms, over a set of training data to make predictions or forecasts. The ensemble of forecasting models may include a first ensemble for forecasting a KPI for entities of a first size and a second ensemble forecasting a KPI for entities of a second size.

One or more embodiments access a features dictionary including additional KPIs and/or engineered features. The system associates the features dictionary with the aggregated dataset to account for external factors.

One or more embodiments described in this Specification and/or recited in the claims may not be included in this General Overview section.

### 2. Key Performance Indicator Forecasting System Architecture

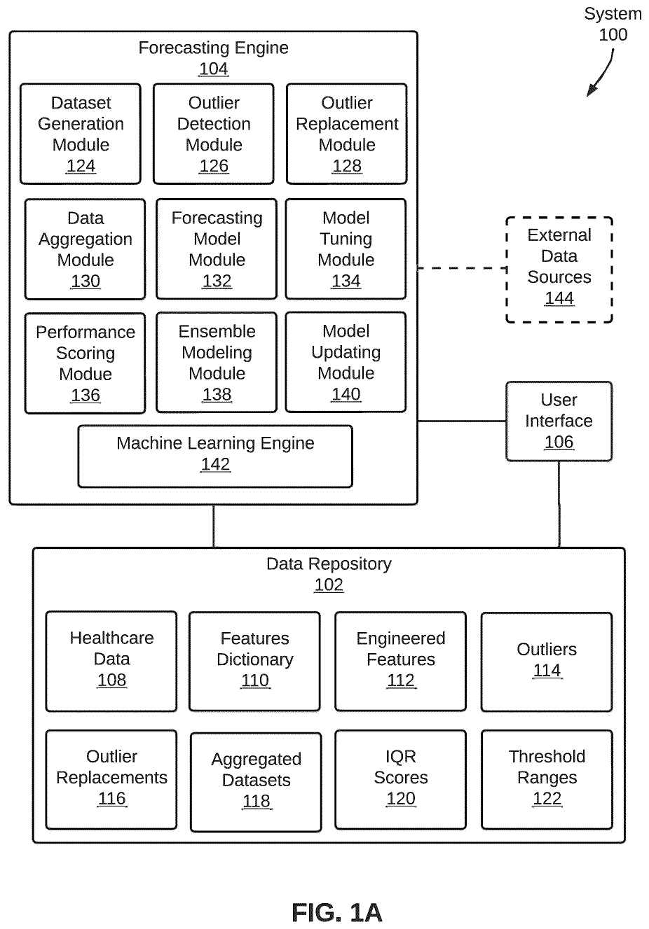

FIG. 1 illustrates a system 100 in accordance with one or more embodiments. As illustrated in FIG. 1, system 100 includes a data repository 102, a forecasting engine 104, and a user interface 106. External data sources 144 are optionally included in the system 100. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the forecasting engine 104. Additionally, or alternatively, a data repository 102 may be implemented or executed on a computing system separate from forecasting engine 104. The data repository 102 may be communicatively coupled to forecasting engine 104 via a direct connection or via a network.

Information describing forecasting engine 104 may be implemented across any of components within the system 100. However, this information is illustrated within the data repository 102 for purposes of clarity and explanation.

In one or more embodiments, the data repository 102 is populated with information from a variety of sources and/or systems. The data repository 102 may be populated with data, such as healthcare data 108, a features dictionary 110, engineered features 112, outliers 114, outlier replacements 116, aggregated datasets 118, IQR scores 120, and threshold ranges 122. Any of this information may be stored in a structured format, e.g., a table.

In one or more embodiments, healthcare data is retrieved from Electronic Health Records (EHR) systems, RCM systems, enterprise resource planning (ERP), patient management systems (PMS), and/or business intelligence (BI) tools. EHR systems, e.g., Epic, Cerner, or Allscripts, contain patient-related data, including clinical workflows, demographics, admissions, and discharge information. EHRs can be queried for time series data such as admissions per month, patient outcomes, or length of stay. EHR systems may have data analytics modules that can generate KPI dashboards. RCM systems manage the lifecycle of patient interactions, providing time series data on claims, reimbursements, denial rates, and payment patterns. ERPs, e.g., Oracle or SAP Healthcare, track operational KPIs, e.g., staff utilization, inventory management, or operational costs, allowing generation of time series data on resources and efficiency. PMS systems track patient-related operational data, such as appointment scheduling, wait times, and readmissions, which can be used to derive operational KPIs. BI platforms, e.g., Tableau, Power BI, or QlikView integrate with healthcare data systems, allowing aggregation, visualization, and analyzing of time series data points.

In one or more embodiments, healthcare data 108 is derived from patient billing, insurance claims, electronic health records (EHRs), and operational systems. Healthcare data may be arranged daily, weekly, monthly, quarterly, or annually. Healthcare data 108 may include data associated with patient demographics, billing and claims, payer, accounts receivable, denials management, payment, charge entry and coding, revenue cycle operation, cost of care services utilization, and pay mix. Patient demographics data includes information about patient age, gender, location, and insurance coverage, e.g., public, private, or self-pay. Billing and claims data includes detailed records of charges submitted to payers, including the date of service, procedure codes (CPT/ICD), billed amounts, and modifiers. Payer data includes data related to insurance companies, including reimbursement rates, payment patterns, and contract details. Accounts receivable includes detailed information about unpaid claims, including outstanding balances, aging categories (e.g., 0-30 days, 31-60 days, 61-90 days), and payment histories. Denials management includes data on claim denials, including reasons for denial, claim types, payer-specific denial rates, and appeal outcomes. Payment data includes records of payments received, including payment amounts, remittance advice, explanation of benefits (EOB), and date of payment. Charge entry and coding includes data related to coding and charge entry for services rendered, including CPT, ICD-10, and HCPCS codes. Revenue cycle operation includes Operational metrics from revenue cycle workflows, such as claim submission times, payment posting times, and staff productivity. Cost of care and service utilization includes data related to the costs of providing services, including physician fees, diagnostic tests, hospital stays, and other resources. Pay mix data includes a breakdown of payer types (e.g., Medicare, Medicaid, private insurance, self-pay) over time.

In one or more embodiments, features dictionary 110 is a structured collection of features that are derived from raw data and used as input variables for training models. The features are transformations or extractions of raw data points that capture relevant patterns or relationships in the dataset. Components of feature dictionary 110 may include Feature Name, Feature Type, Description, Source, Transformation, Time Lag—if applicable, Feature Group. Feature Name is a clear, descriptive name for each feature that represents its purpose or derivation. Feature Type specifies the data type (e.g., numerical, categorical, date, etc.). Description is a detailed explanation of how the feature is derived or what it represents. Source is the original data column(s) or tables from which the feature is derived. Transformation describes the mathematical or logical transformation applied to the raw data to create the feature. Time Lag, for time series data, indicates whether the feature is lagged by a certain period (e.g., one month, one quarter). Feature Group is a logical grouping of related features (e.g., “Financial Features”, “Patient Demographics”).

In one or more embodiments, engineered features 112 are variables created from raw data to enhance the performance of machine learning models. Feature engineering transforms or combines existing data points into features that better represent the underlying patterns in the dataset, improving the model's ability to predict or classify outcomes. Engineered features 112 may include aggregated features, lagged features, rolling/moving statistics, categorical encodings, ratio features, time based features, Boolean features, interaction features, derived features, and cumulative features. Aggregated features are summary statistics calculated over a certain period, such as averages, sums, or counts. Lagged features are previous values of a time-series variable are used as predictors for future values. Rolling/moving statistics are rolling averages, sums, or other statistics over a sliding window of time. Categorical encodings are categorical variables like payer type or procedure codes are transformed into numerical representations using methods such as one-hot encoding or label encoding. Ratio features are ratios between two related variables can reveal important relationships. Time-based features are derived from the date or time of events, such as the month, quarter, or day of the week. Boolean features are binary features that indicate whether a condition is met (True/False). Interaction features are created by combining two or more variables to capture interaction effects. Derived features are custom features that are created through domain-specific transformations or calculations. Cumulative features are features that track cumulative totals over time.

In one or more embodiments, lagged features were conceptualized from the observation that charges posted get converted to payments with a lag or account receivables (AR) of 30-40 days for big government payers, e.g., Medicare. Similarly, for footfall or patients discharged or treated in a month, payment is received post claim clearance with AR of around 2 months.

In one or more embodiments, outliers 114 refer to data points that significantly deviate from the majority of the data, either by being unusually high or low. Outliers can indicate abnormal behaviors or rare events that impact financial or operational performance, such as unexpected claim denials, large payments, or long delays in accounts receivable (A/R).

In one or more embodiments, outlier replacements 116 are values that replace the data points that have been identified as outliers 114. An outlier replacement may be the median of the neighboring data points of the outlier. The neighboring data points may include “M” data points before the outlier and “M” data points after the outlier. If the neighboring data points includes an additional outlier, the additional outlier data point is not used in calculating the median. The additional outlier data point may is removed and an additional data point from the healthcare data is added to the dataset to calculate a median. The additional outlier data point may be replaced by a zero or other variable.

In one or more embodiments, aggregated datasets 118 are datasets associated with the KPIs. Aggregated datasets 118 includes data from the healthcare data 108 that has undergone data preparation. Aggregated datasets 118 include outlier replacements 116 in place of outliers 114. Aggregated datasets 118 may be linked or otherwise associated with engineered features 112 in features dictionary 110.

In one or more embodiments, IQR scores 120 refer to statistical measures used to detect outliers in a dataset. The IQR represents the middle 50% of data points. The first quartile (Q1) of the dataset is the 25^{th} percentile, meaning 25% of the data points fall below this value. The third quartile (Q3) of the dataset is the 75^{th} percentile, meaning 75% of the data points fall below this value. The IQR is calculated as the difference between the third quartile (Q3) and the first quartile (Q1). IQR scores 120 are focused on the central portion of the data and are less affected by extreme values compared to metrics like the range.

In one or more embodiments, threshold ranges 122 refer to lower and upper bounds beyond which data points are considered potential outliers. Threshold ranges 122 are based on IQR scores 120. Thresholds ranges 122 may be modified to increase or lessen strictness.

In one or more embodiments, forecasting engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

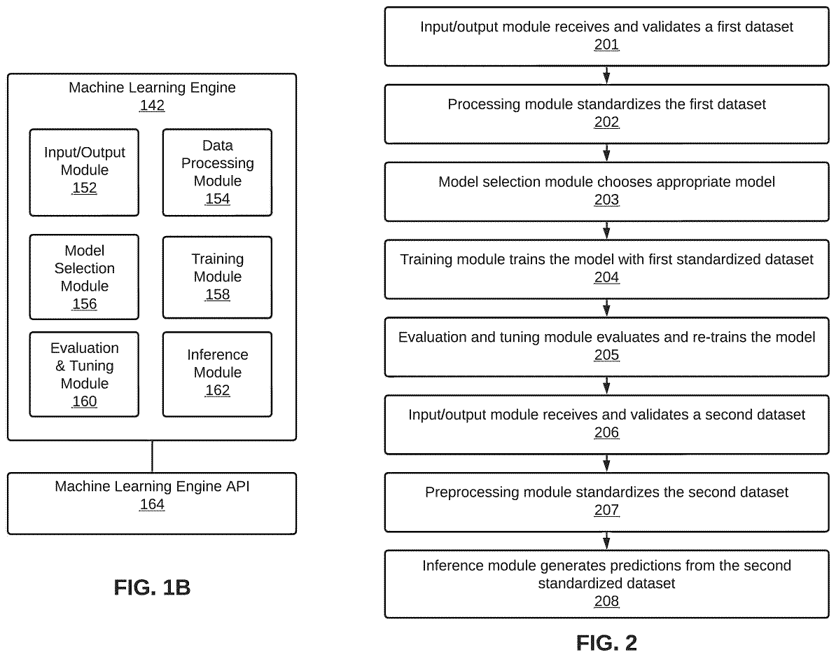

In one or more embodiments, forecasting engine 104 refers to hardware and/or software configured to perform operations described herein for generating datasets for training ensembles of forecasting models to predict KPIs for RCM in healthcare. Examples of operations for generating datasets are described below with reference to FIG. 2. The forecasting engine 104 may include a dataset generation module 124, an outlier detection module 126, an outlier replacement module 128, a dataset aggregation module 130, a forecasting model module 132, a model tuning module 134, a performance scoring module 136, an ensemble modeling module 138, and a model updating module 140.

In one or more embodiment, data extraction module 124 refers to hardware and/or software configured to perform operations described herein for collecting, retrieving, and processing data from various sources to build datasets for training forecasting models 132. Data extraction module 124 automates the process of pulling data from multiple locations, ensuring that data is ready for further transformations or analysis. Data extraction module 126 may be used to extract data from diverse sources like EHRs, claims systems, financial databases, and external Application Programming Interfaces (APIs).

In one or more embodiments, outlier detection module 126 refers to hardware and/or software configured to perform operations described herein for detecting outliers in the healthcare data 108. Outlier detection module 126 may identify and flag data points that deviate significantly from the rest of the dataset. Identifying outliers is essential for improving data quality and model accuracy. Statistical methods, distance-based methods, density-based methods, and machine learning-based methods may be employed by outlier detection module 126 to identify outliers.

In one or more embodiments, outlier detection module 126 employs one or more of the following methods for determining outliers, Z-Score, Boxplot Analysis, Moving Average with Thresholds, Isolation Forest, Local Outlier Factor (LOF), and Visual Inspection. The Z-score measures how far a data point is from the mean in terms of standard deviations. Typically, a Z-score greater than 3 or less than −3 is considered an outlier. Boxplots visually display outliers as points that fall outside the “whiskers,” which represent 1.5 times an IQR. A moving average can smooth out short-term fluctuations and highlight sudden spikes or dips as outliers. Isolation Forest is an anomaly detection algorithm that isolates data points by randomly selecting features and splitting the data. Data points that are more easily isolated are considered outliers. LOF measures the local density of a data point compared to its neighbors. Points with a significantly lower density than their neighbors are classified as outliers. Manual inspection of time-series plots or scatterplots of RCM data can help identify outliers that are not captured by statistical methods.

In one or more embodiments, outlier detection module 126 employs the interquartile range (IQR) method to determine outliers. The IQR method uses the spread between the 25th and 75th percentiles to detect outliers. In an example, data points that fall below Q1−1.5×IQR or above Q3+1.5×IQR are classified as outliers. Using a larger multiplier, e.g., 2.5, increases a strictness of the method, i.e., widens a threshold range, lessening the potential for outliers. Using a smaller multiplier, e.g., 1, decreases the strictness of the method, i.e., tightens a threshold range, increasing the potential for outliers.

In one or more embodiments, outlier replacement module 128 refers to hardware and/or software configured to perform operations described herein for determining replacement values for outliers. Outlier replacement module 128 may employ various replacement strategies including mean/median imputation, IQR capping, mode imputation, linear interpolation, and/or domain-specific imputation. Mean/median imputation replaces outliers with the mean or median of the non-outlier values. Median may be preferred when the data is skewed, as median is less sensitive to extreme values. IQR capping replaces the outliers with the closest value within a pre-defined range, typically within 1.5 times the IQR from the lower or upper quartile. IQR capping approach “caps” outliers, preventing extreme values from distorting the data. Mode imputation replaces outliers with the most frequent value (mode). Mode imputation is useful for categorical variables where outliers can be replaced with the most common category. Linear interpolation replaces outliers with values estimated by interpolating between nearby data points. Linear interpolation is common in time series data where a smooth trend is expected. Domain-specific imputation replaces outliers based on domain-specific rules. For example, claims exceeding a certain threshold may be capped at a regulatory maximum or historical average.

In one or more embodiments, dataset generation module 130 refers to hardware and/or software configured to perform operations described herein for transforming the healthcare data 108 into aggregated datasets 118 for training the forecasting models. Transforms raw data from existing sources, e.g., databases, APIs, CSV files, into a structured dataset. This includes cleaning, normalizing, and enriching the data to prepare the data for analysis.

In one or more embodiments, forecasting model module 132 refers to hardware and/or software configured to perform operations described herein for applying the aggregated datasets 118 to advanced forecasting models for forecasting KPI in healthcare RCM. Each of the forecasting models may be trained on the same or different subsets of the data.

In one or more embodiments, forecasting model module 132 uses Seasonal Autoregressive Integrated Moving Average (SARIMA) as a modeling technique. SARIMA extends the Autoregressive Integrated Moving Average (ARIMA) model by adding components that handle seasonality in the data. SARMA is especially useful when patterns repeat at regular intervals, e.g., daily, monthly, or yearly. Components of SARIMA may include seasonal autoregressive (SAR), seasonal differencing (D), and seasonal moving average (SMA) terms. SARIMA is highly effective in predicting recurring financial or operational trends, e.g., monthly revenue cycles or seasonal patient admissions in healthcare. SARIMA captures both trend and seasonality.

In one or more embodiments, forecasting model module 132 uses Holt-Winter's Exponential Smoothing (HWES). HWES is a method of exponential smoothing that models data with both a trend and seasonality. HWES has two variations, additive and multiplicative, depending on the nature of the trend and seasonality. HWES may be used for forecasting with short-to medium-term seasonal data, i.e., daily patient volumes or monthly billing amounts. HWES is simple and effective for capturing seasonal trends in data.

In one or more embodiments, forecasting module 132 uses Trigonometric Box-Cox Transformation ARMA Errors Trend Seasonality (TBATS) as a modeling technique. TBATS is a flexible state-space model designed to handle complex seasonal patterns, including non-integer and multiple seasonalities, and long seasonal cycles. TBATS is useful when the data has multiple seasonal patterns, i.e., daily and yearly fluctuations in patient flows or sales. TBATS handles multiple and non-integer seasonalities. TBATS is useful for forecasting data with multiple time scales, e.g., weekly cycles and annual trends. SARIMAX extends SARIMA by incorporating exogenous variables, i.e., independent variables, into the model. This allows the model to include external factors that could influence the forecast, e.g., policy changes or economic indicators. SARIMAX can be used in scenarios where external factors, e.g., payer policies, market dynamics, or seasonal factors, influence outcomes like revenue cycles, claim approval rates, or cash collections. SARIMAX incorporates external influences, making predictions more accurate.

In one or more embodiments, forecasting module 132 uses Vector Autoregressive Moving Average (VARMA) as a modeling technique. VARMA models the dynamic relationship between multiple time series by extending Autoregressive Moving Average (ARMA) to handle multivariate data. VARMA captures the linear interdependencies between several variables. VARMA is useful for forecasting interdependent variables, i.e., revenue and claim denial rates, or patient admissions and staff scheduling, where multiple time series are related. VARMA models multiple time series simultaneously.

In one or more embodiments, forecasting module 132 uses Vector Autoregressive Moving Average with eXogenous Regressors (VARMAX) as a modeling technique. VARMAX extends VARMA by allowing exogenous variables, which makes VARMAX more flexible for capturing relationships between several time series and external factors. VARMAX is useful for forecasting scenarios with multiple interdependent variables and the influence of external factors, e.g., healthcare outcomes influenced by government policies or insurance claims affected by economic conditions. VARMAX incorporates both interdependent variables and external factors.

In one or more embodiments, forecasting model module 132 uses Prophet as a modeling technique. Prophet is an open-source tool, developed by Facebook, designed for easy and fast forecasting with seasonal and trend components. Prophet automatically detects change points and adjusts predictions. Prophet may be used for time series data that shows seasonality, holidays, or other irregular patterns.

In one or more embodiments, model tuning module 134 refers to hardware and/or software configured to perform operations described herein for optimizing the performance of forecasting models by adjusting hyperparameters. The hyperparameters control how the model learns from the data. Proper tuning can significantly improve a model's accuracy, robustness, and generalization to new data. Model tuning module 134 is to find the best combination of hyperparameters for a given model. Hyperparameters may include learning rate, number of estimators, ARIMA/SARIMA components, e.g., p, d, q, and seasonal parameters.

In one or more embodiments, tuning strategies employed by model tuning module 134 include, grid search, random search, Bayesian optimization, and/or genetic algorithms. Grid search is a brute-force method that tries all possible combinations of hyperparameters within a specified range. Random search randomly selects combinations of hyperparameters to explore a larger space with fewer evaluations. Bayesian optimization uses past evaluations to choose the next set of hyperparameters, focusing on the most promising regions of the hyperparameter space. Genetic algorithms, inspired by natural selection, evolve hyperparameter configurations over multiple generations.

In one or more embodiments, model tuning module 134 employs cross-validation, e.g., k-fold cross-validation, walk forward validation, to assess the performance of each hyperparameter setting. Cross-validation splits the data into training and validation sets multiple times to ensure the model's performance is stable across different subsets of the data.

In one or more embodiments, performance scoring module 136 refers to hardware and/or software configured to perform operation described herein for evaluating the performance of models based on specific metrics. Performance scoring module 136 may employ various metrics to evaluate performance. Performance metrics evaluated by performance scoring module 136 may include regression metrics, classification metrics, time series forecasting metrics. Regression metrics include mean absolute percentage error (MAPE), mean squared error (MSE), root mean squared error (RMSE), and mean absolute error (MAE). Classification metrics include accuracy, precision, recall, F1-score, and area under curve (AUC) for ROC. Time series forecasting metrics include MAPE, RMSE, and MAE, mean percentage error (MPE), and mean forecast error (MFE).

In one or more embodiments, ensemble modeling module 138 refers to hardware and/or software configured to perform operations described herein for combining predictions of multiple individual models to improve overall predictive accuracy, robustness, and generalization. Ensemble techniques aggregate the strengths of various models, mitigating the weaknesses of any single model by leveraging diverse methodologies.

In one or more embodiments, ensemble modeling module 138 employs various types of ensemble methods including bagging, boosting, stacking, and voting. Bagging, also referred to as bootstrap aggregating, generates multiple versions of a model using random subsets of data (with replacement) and averages the predictions. Boosting sequentially trains models, where each new model corrects the errors of the previous one. The final prediction is a weighted sum of the predictions. Stacking combines the predictions of multiple models, called base models, through a meta-model, i.e., a higher-level model, that learns how to best combine the base model predictions. Voting aggregates the predictions of several models through majority voting or averaging.

In one or more embodiments, a strength of ensemble modeling is the diversity of the component models. Models that make different kinds of errors can complement each other. Ensemble modeling module 138 ensures that the ensemble contains models with different assumptions, learning mechanisms, and/or hyperparameters to avoid correlated errors. Some ensemble modeling methods, e.g., weighted voting or stacking, allow different models to be assigned weights based on the accuracy or reliability of the models. More accurate models contribute more to the final prediction. Ensemble modeling module 138 may include processes for selecting the best individual models to include in the ensemble and for tuning hyperparameters, e.g., number of base models, learning rates, model weights, of the models in the ensembles.

In one or more embodiments, model updating module 140 refers to hardware and/or software configured to perform operations described herein for managing the continuous improvement and maintenance of the machine learning models. Model updating module 140 automates the process of updating, re-training, and deploying models based on new data, changing business requirements, or detected performance issues. Model updating module 140 ensures that models remain accurate, relevant, and robust over time.

In one or more embodiments, model updating module 140 monitors the performance of deployed models in real-time, as provided by performance scoring module 136, checking for performance degradation or anomalies. Model updating module 140 can detect issues like concept drift, where the underlying data distribution shifts, causing a model to become less accurate.

In one or more embodiments, model updating module 140 performs updates when trigger conditions are satisfied. Triggers for updating the forecasting models may include performance degradation, scheduled updates, data availability, manual triggers.

Performance degradation includes triggering an update or retraining process when a model's performance falls below a pre-defined threshold. Models may be retrained on a regular schedule, e.g., weekly or monthly, to incorporate new data and ensure continued performance. Model updating module 140 can be triggered when a significant amount of new data is available, e.g., new customer data, financial data, or healthcare records. Data scientists or engineers may manually trigger an update when changes in business objectives or external conditions, e.g., policy changes in healthcare, are observed.

In one or more embodiments, model updating module 140 re-trains models using the latest available data. This can include incremental learning or full retraining. With incremental learning, new data can be used to incrementally update a model without needing to retrain from scratch. For some models, retraining on the full dataset might be required to refresh predictions with the latest trends. Model updating module 140 manages the data pipeline, ensuring that the data used for retraining is cleaned, processed, and aligned with previous versions of the dataset, e.g., handling schema changes, new features, or missing values.

In one or more embodiments, forecasting engine 104 includes machine learning engine 142. Machine learning engine 142 refers to hardware and/or software configured to perform the operations described herein for training and applying machine learning models. The structure and function of machine learning engine 142 will be described below in detail with reference to FIGS. 1B and 2.

In one or more embodiments, user interface 106 refers to hardware and/or software configured to facilitate communications between a user and forecasting engine 104. User interface 106 renders user interface elements and receives input via user interface elements. Examples of interfaces include a graphical user interface, a command line interface, a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language or XML User Interface Language. The layout of user interface elements is specified in a style sheet language, such as Cascading Style Sheets. Alternatively, user interface 106 is specified in one or more other languages, such as Java, C, or C++.

In one or more embodiments, external data sources 144 refer to data that comes from outside an organization's own systems and infrastructure. External data sources 144 provide valuable additional information to enhance forecasting. External data sources may help improve model accuracy and provide insights that internal data alone may not capture.

In one or more embodiments, external data sources 142 include public datasets, third-party vendors, weather and environmental data, and social media and web data. Government agencies often publish open datasets related to healthcare, economy, finance, demographics, e.g., Centers for Medicare & Medicaid Services (CMS) data, Census Data, World Bank & IMF Data forecasts. Market research firms, e.g., Gartner, Forrester, Nielsen, provide market trends, customer behavior data, and competitive landscape insights. Healthcare analytics companies specialize in healthcare data, e.g., IQVIA, and provide real-world data on treatment patterns, patient outcomes, and financial performance. Insurance claims data include databases that provide access to aggregated insurance claim statistics, denial rates, and reimbursement trends across various payers. Weather data providers, e.g., National Oceanic and Atmospheric Administration, AccuWeather, Weather.com, offer real-time and historical weather information. Environmental data includes factors like air quality, natural disasters, and temperature. Social media and web data includes tools that can extract data from social media platforms like Twitter, Facebook, or LinkedIn to gauge public opinion, product sentiment, or trends in consumer behavior. Organizations may use web scraping techniques to gather data from websites.

In one or more embodiments, external data sources 144 include industry-specific data sources, geospatial data, industry benchmarks and competitor data, demographic and psychographic data, and pay mix data. For healthcare organizations, data sources such as EHRs vendors, patient surveys, and clinical trial data can provide valuable insights. Energy providers, e.g., Energy Information Administration, provide data on energy consumption, prices, and production. Geographic Information Systems (GIS) data sources like Google Maps, OpenStreetMap, or ESRI provide geospatial data that can help with location-based analyses, such as market expansion or supply chain optimization. Competitive intelligence tools like SimilarWeb, Ahrefs, or SEMrush provide information on competitors'web traffic, marketing strategies, and keyword performance. Various reports provide benchmarking data that allows organizations to compare their performance against industry standards. These reports can be obtained from industry associations or consultancy firms. Companies like Experian, Acxiom, or Neustar offer detailed demographic, psychographic, and behavioral data, which can be used for customer segmentation and targeted marketing. Polling organizations, e.g., Pew Research or Gallup, provide insights into public opinions, consumer preferences, and societal trends. External data on the payer mix may influence revenue cycle models in healthcare.

### 3. Machine Learning Architecture

FIG. 1 illustrates a machine learning engine 142 in accordance with one or more embodiments. As illustrated in FIG. 1, machine learning engine 142 includes input/output module 152, data preprocessing module 154, model selection module 156, training module 158, evaluation and tuning module 160, and inference module 162.

In accordance with an embodiment, input/output module 152 serves as the primary interface for data entering and exiting the system, managing the flow and integrity of data. This module may accommodate a wide range of data sources and formats to facilitate integration and communication within the machine learning architecture.

In an embodiment, an input handler within input/output module 152 includes a data ingestion framework capable of interfacing with various data sources, such as databases, APIs, file systems, and real-time data streams. This framework is equipped with functionalities to handle different data formats (e.g., CSV, JSON, XML) and efficiently manage large volumes of data. It includes mechanisms for batch and real-time data processing that enable the input/output module 152 to be versatile in different operational contexts, whether processing historical datasets or streaming data.

In accordance with an embodiment, input/output module 152 manages data integrity and quality as it enters the system by incorporating initial checks and validations. These checks and validations ensure that incoming data meets predefined quality standards, like checking for missing values, ensuring consistency in data formats, and verifying data ranges and types. This proactive approach to data quality minimizes potential errors and inconsistencies in later stages of the machine learning process.

In an embodiment, an output handler within input/output module 152 includes an output framework designed to handle the distribution and exportation of outputs, predictions, or insights. Using the output framework, input/output module 152 formats these outputs into user-friendly and accessible formats, such as reports, visualizations, or data files compatible with other systems. Input/output module 152 also ensures secure and efficient transmission of these outputs to end-users or other systems in an embodiment and may employ encryption and secure data transfer protocols to maintain data confidentiality.

In accordance with an embodiment, data preprocessing module 154 transforms data into a format suitable for use by other modules in machine learning engine 142. For example, data preprocessing module 154 may transform raw data into a normalized or standardized format suitable for training ML models and for processing new data inputs for inference. In an embodiment, data preprocessing module 154 acts as a bridge between the raw data sources and the analytical capabilities of machine learning engine 142.

In an embodiment, data preprocessing module 154 begins by implementing a series of preprocessing steps to clean, normalize, and/or standardize the data. This involves handling a variety of anomalies, such as managing unexpected data elements, recognizing inconsistencies, or dealing with missing values. Some of these anomalies can be addressed through methods like imputation or removal of incomplete records, depending on the nature and volume of the missing data. Data preprocessing module 154 may be configured to handle anomalies in different ways depending on context. Data preprocessing module 154 also handles the normalization of numerical data in preparation for use with models sensitive to the scale of the data, like neural networks and distance-based algorithms. Normalization techniques, such as min-max scaling or z-score standardization, may be applied to bring numerical features to a common scale, enhancing the model's ability to learn effectively.

In an embodiment, data preprocessing module 154 includes a feature encoding framework that ensures categorical variables are transformed into a format that can be easily interpreted by machine learning algorithms. Techniques like one-hot encoding or label encoding may be employed to convert categorical data into numerical values, making them suitable for analysis. The module may also include feature selection mechanisms, where redundant or irrelevant features are identified and removed, thereby increasing the efficiency and performance of the model.

In accordance with an embodiment, when data preprocessing module 154 processes new data for inference, data preprocessing module 154 replicates the same preprocessing steps to ensure consistency with the training data format. This helps to avoid discrepancies between the training data format and the inference data format, thereby reducing the likelihood of inaccurate or invalid model predictions.

In an embodiment, model selection module 156 includes logic for determining the most suitable algorithm or model architecture for a given dataset and problem. This module operates in part by analyzing the characteristics of the input data, such as its dimensionality, distribution, and the type of problem (classification, regression, clustering, etc.).

In an embodiment, model selection module 156 employs a variety of statistical and analytical techniques to understand data patterns, identify potential correlations, and assess the complexity of the task. Based on this analysis, it then matches the data characteristics with the strengths and weaknesses of various available models. This can range from simple linear models for less complex problems to sophisticated deep learning architectures for tasks requiring feature extraction and high-level pattern recognition, such as image and speech recognition.

In an embodiment, model selection module 156 utilizes techniques from the field of Automated Machine Learning (AutoML). AutoML systems automate the process of model selection by rapidly prototyping and evaluating multiple models. They use techniques like Bayesian optimization, genetic algorithms, or reinforcement learning to explore the model space efficiently. Model selection module 156 may use these techniques to evaluate each candidate model based on performance metrics relevant to the task. For example, accuracy, precision, recall, or F1 score may be used for classification tasks and mean squared error metrics may be used for regression tasks. Accuracy measures the proportion of correct predictions (both positive and negative). Precision measures the proportion of actual positives among the predicted positive cases. Recall (also known as sensitivity) evaluates how well the model identifies actual positives. F1 Score is a single metric that accounts for both false positives and false negatives. The MSE metric may be used for regression tasks. MSE measures the average squared difference between the actual and predicted values, providing an indication of the model's accuracy. A lower MSE may indicate a model's greater accuracy in predicting values, as it represents a smaller average discrepancy between the actual and predicted values.

In accordance with an embodiment, model selection module 156 also considers computational efficiency and resource constraints. This is meant to help ensure the selected model is both accurate and practical in terms of computational and time requirements. In an embodiment, certain features of model selection module 156 are configurable such as a configured bias toward (or against) computational efficiency.

In accordance with an embodiment, training module 158 manages the ‘learning’ process of ML models by implementing various learning algorithms that enable models to identify patterns and make predictions or decisions based on input data. In an embodiment, the training process begins with the preparation of the dataset after preprocessing; this involves splitting the data into training and validation sets. The training set is used to teach the model, while the validation set is used to evaluate its performance and adjust parameters accordingly.

Training module 158 handles the iterative process of feeding the training data into the model, adjusting the model's internal parameters (like weights in neural networks) through backpropagation and optimization algorithms, such as stochastic gradient descent or other algorithms providing similarly useful results.

In accordance with an embodiment, training module 158 manages overfitting, where a model learns the training data too well, including its noise and outliers, at the expense of its ability to generalize to new data. Techniques such as regularization, dropout (in neural networks), and early stopping are implemented to mitigate this. Additionally, the module employs various techniques for hyperparameter tuning; this involves adjusting model parameters that are not directly learned from the training process, such as learning rate, the number of layers in a neural network, or the number of trees in a random forest.

In an embodiment, training module 158 includes logic to handle different types of data and learning tasks. For instance, it includes different training routines for supervised learning (where the training data comes with labels) and unsupervised learning (without labeled data). In the case of deep learning models, training module 158 also manages the complexities of training neural networks that include initializing network weights, choosing activation functions, and setting up neural network layers.

In an embodiment, evaluation and tuning module 160 incorporates dynamic feedback mechanisms and facilitates continuous model evolution to help ensure the system's relevance and accuracy as the data landscape changes. Evaluation and tuning module 160 conducts a detailed evaluation of a model's performance. This process involves using statistical methods and a variety of performance metrics to analyze the model's predictions against a validation dataset. The validation dataset, distinct from the training set, is instrumental in assessing the model's predictive accuracy and its capacity to generalize beyond the training data. The module's algorithms meticulously dissect the model's output, uncovering biases, variances, and the overall effectiveness of the model in capturing the underlying patterns of the data.

In an embodiment, evaluation and tuning module 160 performs continuous model tuning by using hyperparameter optimization. Evaluation and tuning module 160 performs an exploration of the hyperparameter space using algorithms, such as grid search, random search, or more sophisticated methods like Bayesian optimization. Evaluation and tuning module 160 uses these algorithms to iteratively adjust and refine the model's hyperparameters—settings that govern the model's learning process but are not directly learned from the data—to enhance the model's performance. This tuning process helps to balance the model's complexity with its ability to generalize and attempts to avoid the pitfalls of underfitting or overfitting.

In an embodiment, evaluation and tuning module 160 integrates data feedback and updates the model. Evaluation and tuning module 160 actively collects feedback from the model's real-world applications, an indicator of the model's performance in practical scenarios. Such feedback can come from various sources depending on the nature of the application. For example, in a user-centric application like a recommendation system, feedback might comprise user interactions, preferences, and responses. In other contexts, such as predicting events, it might involve analyzing the model's prediction errors, misclassifications, or other performance metrics in live environments.

In an embodiment, feedback integration logic within evaluation and tuning module 160 integrates this feedback using a process of assimilating new data patterns, user interactions, and error trends into the system's knowledge base. The feedback integration logic uses this information to identify shifts in data trends or emergent patterns that were not present or inadequately represented in the original training dataset. Based on this analysis, the module triggers a retraining or updating cycle for the model. If the feedback suggests minor deviations or incremental changes in data patterns, the feedback integration logic may employ incremental learning strategies, fine-tuning the model with the new data while retaining its previously learned knowledge. In cases where the feedback indicates significant shifts or the emergence of new patterns, a more comprehensive model updating process may be initiated. This process might involve revisiting the model selection process, re-evaluating the suitability of the current model architecture, and/or potentially exploring alternative models or configurations that are more attuned to the new data.

In accordance with an embodiment, throughout this iterative process of feedback integration and model updating, evaluation and tuning module 160 employs version control mechanisms to track changes, modifications, and the evolution of the model, facilitating transparency and allowing for rollback if necessary. This continuous learning and adaptation cycle, driven by real-world data and feedback, helps to endure the model's ongoing effectiveness, relevance, and accuracy.

In an embodiment, inference module 162 transforms data raw data into actionable, precise, and contextually relevant predictions. In addition to processing and applying a trained model to new data, inference module 162 may also include post-processing logic that refines the raw outputs of the model into meaningful insights.

In an embodiment, inference module 162 includes classification logic that takes the probabilistic outputs of the model and converts them into definitive class labels. This process involves an analytical interpretation of the probability distribution for each class. For example, in binary classification, the classification logic may identify the class with a probability above a certain threshold, but classification logic may also consider the relative probability distribution between classes to create a more nuanced and accurate classification.

In an embodiment, inference module 162 transforms the outputs of a trained model into definitive classifications. Inference module 162 employs the underlying model as a tool to generate probabilistic outputs for each potential class. It then engages in an interpretative process to convert these probabilities into concrete class labels.

In an embodiment, when inference module 162 receives the probabilistic outputs from the model, it analyzes these probabilities to determine how they are distributed across some or every potential class. If the highest probability is not significantly greater than the others, inference module 162 may determine that there is ambiguity or interpret this as a lack of confidence displayed by the model.

In an embodiment, inference module 162 uses thresholding techniques for applications where making a definitive decision based on the highest probability might not suffice due to the critical nature of the decision. In such cases, inference module 162 assesses if the highest probability surpasses a certain confidence threshold that is predetermined based on the specific requirements of the application. If the probabilities do not meet this threshold, inference module 162 may flag the result as uncertain or defer the decision to a human expert. Inference module 162 dynamically adjusts the decision thresholds based on the sensitivity and specificity requirements of the application, subject to calibration for balancing the trade-offs between false positives and false negatives.

In accordance with an embodiment, inference module 162 contextualizes the probability distribution against the backdrop of the specific application. This involves a comparative analysis, especially in instances where multiple classes have similar probability scores, to deduce the most plausible classification. In an embodiment, inference module 162 may incorporate additional decision-making rules or contextual information to guide this analysis, ensuring that the classification aligns with the practical and contextual nuances of the application.

In regression models, where the outputs are continuous values, inference module 162 may engage in a detailed scaling process in an embodiment. Outputs, often normalized or standardized during training for optimal model performance, are rescaled back to their original range. This rescaling involves recalibration of the output values using the original data's statistical parameters, such as mean and standard deviation, ensuring that the predictions are meaningful and comparable to the real-world scales they represent.

In an embodiment, inference module 162 incorporates domain-specific adjustments into its post-processing routine. This involves tailoring the model's output to align with specific industry knowledge or contextual information. For example, in financial forecasting, inference module 162 may adjust predictions based on current market trends, economic indicators, or recent significant events, ensuring that the outputs are both statistically accurate and practically relevant.

In an embodiment, inference module 162 includes logic to handle uncertainty and ambiguity in the model's predictions. In cases where inference module 162 outputs a measure of uncertainty, such as in Bayesian inference models, inference module 162 interprets these uncertainty measures by converting probabilistic distributions or confidence intervals into a format that can be easily understood and acted upon. This provides users with both a prediction and an insight into the confidence level of that prediction. In an embodiment, inference module 162 includes mechanisms for involving human oversight or integrating the instance into a feedback loop for subsequent analysis and model refinement.

In an embodiment, inference module 162 formats the final predictions for end-user consumption. Predictions are converted into visualizations, user-friendly reports, or interactive interfaces. In some systems, like recommendation engines, inference module 162 also integrates feedback mechanisms, where user responses to the predictions are used to continually refine and improve the model, creating a dynamic, self-improving system.

### 4. Machine Learning Engine Operation

FIG. 2 illustrates the operation of a machine learning engine in one or more embodiments. In an embodiment, input/output module 152 receives a dataset intended for training (Operation 201). This data can originate from diverse sources, like databases or real-time data streams, and in varied formats, such as CSV, JSON, or XML. Input/output module 152 assesses and validates the data, ensuring its integrity by checking for consistency, data ranges, and types.

In an embodiment, training data is passed to data preprocessing module 154. Here, the data undergoes a series of transformations to standardize and clean it, making it suitable for training ML models (Operation 202). This involves normalizing numerical data, encoding categorical variables, and handling missing values through techniques like imputation.

In an embodiment, prepared data from the data preprocessing module 154 is then fed into model selection module 156 (Operation 203). This module analyzes the characteristics of the processed data, such as dimensionality and distribution, and selects the most appropriate model architecture for the given dataset and problem. It employs statistical and analytical techniques to match the data with an optimal model, ranging from simpler models for less complex tasks to more advanced architectures for intricate tasks.

In an embodiment, training module 158 trains the selected model with the prepared dataset (Operation 204). It implements learning algorithms to adjust the model's internal parameters, optimizing them to identify patterns and relationships in the training data. Training module 158 also addresses the challenge of overfitting by implementing techniques, like regularization and early stopping, ensuring the model's generalizability.

In an embodiment, evaluation and tuning module 160 evaluates the trained model's performance using the validation dataset (Operation 205). Evaluation and tuning module 160 applies various metrics to assess predictive accuracy and generalization capabilities. It then tunes the model by adjusting hyperparameters, and if needed, incorporates feedback from the model's initial deployments, retraining the model with new data patterns identified from the feedback.

In an embodiment, input/output module 152 receives a dataset intended for inference. Input/output module 152 assesses and validates the data (Operation 206).

In an embodiment, data preprocessing module 154 receives the validated dataset intended for inference (Operation 207). Data preprocessing module 154 ensures that the data format used in training is replicated for the new inference data, maintaining consistency and accuracy for the model's predictions.

In an embodiment, inference module 162 processes the new data set intended for inference, using the trained and tuned model (Operation 208). It applies the model to this data, generating raw probabilistic outputs for predictions. Inference module 162 then executes a series of post-processing steps on these outputs, such as converting probabilities to class labels in classification tasks or rescaling values in regression tasks. It contextualizes the outputs as per the application's requirements, handling any uncertainty in predictions and formatting the final outputs for end-user consumption or integration into larger systems.

In an embodiment, machine learning engine API 164 allows for applications to leverage machine learning engine 142. In an embodiment, machine learning engine API 164 may be built on a RESTful architecture and offer stateless interactions over standard HTTP/HTTPS protocols. Machine learning engine API 164 may feature a variety of endpoints, each tailored to a specific function within machine learning engine 142. In an embodiment, endpoints such as /submitData facilitate the submission of new data for processing, while /retrieveResults is designed for fetching the outcomes of data analysis or model predictions. The MLE API may also include endpoints like /updateModel for model modifications and /trainModel to initiate training with new datasets.

In an embodiment, machine learning engine API 164 is equipped to support SOAP-based interactions. This extension involves defining a WSDL (Web Services Description Language) document that outlines the API's operations and the structure of request and response messages. In an embodiment, machine learning engine API 164 supports various data formats and communication styles. In an embodiment, machine learning engine API 164 endpoints may handle requests in JSON format or any other suitable format. For example, machine learning engine API 164 may process XML, and it may also be engineered to handle more compact and efficient data formats, such as Protocol Buffers or Avro, for use in bandwidth-limited scenarios.

In an embodiment, machine learning engine API 164 is designed to integrate WebSocket technology for applications necessitating real-time data processing and immediate feedback. This integration enables a continuous, bi-directional communication channel for a dynamic and interactive data exchange between the application and machine learning engine 142.

### 5. Generative Models

A generative model is a machine learning model that is capable of generating new data instances based on the data used to train the model. A generative model may be referred to as a “generative artificial intelligence (AI) model. ” Generative models learn the underlying distribution of the training data, enabling them to produce new instances of data that share properties with the original dataset. This capability makes them particularly useful in a variety of applications, including image and voice generation, text synthesis, and more sophisticated tasks like unsupervised learning, semi-supervised learning, and domain adaptation.

One type of generative model is a LLM. LLMs are designed to understand, generate, and interpret human language by processing extensive collections of data. The foundational architecture behind LLMs is the transformer network, a type of neural network that excels in handling sequential data such as text. Unlike architectures, such as recurrent neural networks (RNNs) or long short-term memory networks (LSTMs), transformers do not process data in order. Instead, they leverage parallel processing to analyze entire text sequences simultaneously, significantly improving efficiency and reducing training times.

In an embodiment, a mechanism that enables transformers to handle complex language tasks is self-attention. This mechanism allows the model to weigh the importance of different words within a sentence or sequence regardless of their position. For instance, in processing the phrase “The cat sat on the mat,” the model can directly associate “cat” with “mat” without having to process the intermediate words sequentially. This ability to understand the context and relationships between words in a sentence is what makes transformer networks adept at language tasks. The self-attention mechanism assigns scores to relationships between words, highlighting the most relevant connections, so the model can focus on the most informative parts of the text.

In accordance with one or more embodiments, transformers are composed of multiple layers containing a multi-head, self-attention mechanism and a position-wise, feed-forward network. Within the architecture of transformer models, the multi-head, self-attention mechanism and position-wise, feed-forward network function in concert to process input data. The multi-head, self-attention mechanism is designed to enable parallel processing of input sequences, allowing the model to simultaneously evaluate the importance of different segments of the input relative to each other. This mechanism operates by generating multiple sets of query, key, and value vectors for each element in the input sequence through linear transformation. The relevance of each element to every other element is calculated using a scaled dot-product attention function that computes the attention scores by taking the dot product of the query vector with the key vectors, dividing each by the square root of the dimension of the key vectors to scale the scores, then applying a softmax function to obtain the weights for the value vectors. The scaled dot-product attention function is applied independently by each head in the multi-head self-attention mechanism. The outputs of these heads are then concatenated and linearly transformed, allowing the model to capture information from different representation subspaces.

In accordance with one or more embodiments, following the multi-head, self-attention mechanism is the position-wise, feed-forward network. This component comprises two linear transformations with a non-linear activation function in between. Each element of the input sequence, now enriched with context by the self-attention mechanism, is processed independently through the same feed-forward network. The first linear transformation increases the dimensionality of the input, allowing for a richer representation space. The non-linear activation function introduces the capability to capture non-linear relationships within the data. The second linear transformation then reduces the dimensionality back to that of the model's hidden layers, preparing the output for either further processing by subsequent layers or final output generation. This sequence of operations is applied to each position in the sequence, so the model can learn complex patterns across different parts of the input data without relying on the sequential processing inherent to previous architectures, such as RNNs or LSTMs.

In accordance with one or more embodiments, integrating these components within the transformer architecture facilitates the model's ability to understand and generate human language by leveraging both the global context provided by the self-attention mechanism and the local, position-specific transformations applied by the feed-forward networks. Through the repetitive stacking of layers, transformers achieve a depth of representation that allows for the processing of linguistic information across varying levels of complexity.

In accordance with one or more embodiments, input/output module 152, when used for LLMs, handles textual data, converting input text into a format that the model can process. This typically involves tokenization, where the text is broken down into manageable pieces, such as words or subwords, and then converted into numerical representations. These representations, or embeddings, capture semantic information about the text that is then fed into the model for processing. The output from the model is converted from numerical form back into human-readable text, following the generation of predictions or responses.

In accordance with one or more embodiments, data preprocessing module 154 in the context of LLMs may include steps such as normalization, where the text is converted to a uniform case and punctuation is standardized. This process ensures that the model treats similar words or symbols consistently, reducing the complexity of the input space. Additionally, techniques such as sentence segmentation may be applied to manage longer texts, enabling the model to process information in chunks that align with natural language structures.

In accordance with one or more embodiments, model selection module 156, when used for LLMs involves choosing a specific architecture and configuration that is best suited to the task at hand. This decision is based on various factors, such as the size of the available training data, the complexity of the language tasks to be performed, and computational resource constraints. Models may vary in size from millions to billions of parameters, with larger models generally capable of more nuanced language understanding and generation but requiring significantly more computational power to train and operate.

In accordance with one or more embodiments, training module 158, when used for LLMs, is configured to adjust the model's parameters through exposure to training data. This process utilizes optimization algorithms, such as stochastic gradient descent, to minimize the difference between the model's predictions and the actual desired outputs. The training process is computationally intensive, often requiring specialized hardware such as GPUs (Graphics Processing Units) or TPUs (Tensor Processing Units) to manage the large volumes of data and the complexity of the model calculations. During training, techniques, such as dropout and layer normalization, are used to improve model generalization and prevent overfitting (i.e., when a model learns the detail and noise in the training data to the extent that it negatively impacts the model's performance on new data).

In accordance with one or more embodiments, evaluation and tuning module 160 assesses the performance of LLMs using metrics such as perplexity, accuracy, and F1 score, depending on the specific language tasks. Evaluation may involve comparing the model's output against a set of labeled validation data, providing insight into how well the model has learned to perform tasks, such as text classification, question answering, or text generation. Tuning involves adjusting model parameters or training strategies based on evaluation outcomes to improve performance. This may include hyperparameter tuning, where parameters that govern the training process, such as learning rate or batch size, are adjusted.

In accordance with one or more embodiments, inference module 162, in the context of LLMs, is responsible for generating predictions or responses based on new, unseen data. This process involves feeding the input data through the trained model to produce an output. Inference can be used for a variety of applications, including translating text, generating human-like responses in a chatbot, or summarizing articles.

Another type of generative model is a large multimodal model (LMM). A large multimodal model is an advanced machine learning model capable of processing and generating data across multiple modalities, such as text, images, audio, and video. These models integrate diverse datasets during training to learn the underlying distribution of different data types, enabling them to produce outputs that reflect a comprehensive understanding of the input data. These models can be used for applications such as image captioning, text-to-image generation, image-to-text generation, visual question answering, and more, where understanding the relationship between different data types is crucial. By leveraging diverse datasets during training, large multimodal models learn to create coherent and contextually relevant outputs across various modalities, enhancing their utility in complex, real-world scenarios.

The architecture of large multimodal models combines elements from different neural network designs to handle diverse data types effectively. For example, convolutional neural networks (CNNs) are often used for processing visual data, while transformer networks handle textual data, enabling the model to extract and synthesize features from both images and text.

This integration results in outputs that accurately represent the input data, reflecting a deep understanding of both modalities. The transformer architecture, known for its ability to manage sequential data, is frequently adapted to work alongside CNNs, allowing these models to benefit from the strengths of each neural network type.

In at least some instances, the self-attention mechanism, a cornerstone of transformer networks, is integral to the functioning of large multimodal models. It enables the model to weigh the importance of different elements within an input sequence, regardless of their position, allowing it to capture intricate relationships between various data types. For example, in an image captioning task, the model can associate specific visual features with corresponding descriptive text, enhancing the coherence and accuracy of the generated captions. By assigning scores to relationships between elements, the self-attention mechanism highlights the most relevant connections, enabling the model to focus on the most informative parts of the input data and perform complex multimodal tasks effectively.

In large multimodal models, data preprocessing is a step that ensures the input data is in a suitable format for the model to process. This involves tasks such as tokenization for text data, where the text is broken down into manageable pieces, and feature extraction for image data, where key visual elements are identified and encoded. By standardizing and normalizing different data types, preprocessing reduces the complexity of the input space, enabling the model to treat similar elements consistently. Effective preprocessing is essential for the model to integrate information from various modalities and produce accurate, meaningful outputs.

Training large multimodal models involves optimizing their parameters through exposure to diverse datasets that include paired data from different modalities. This computationally intensive process often requires specialized hardware like GPUs or TPUs to manage the large volumes of data and the complexity of the model calculations. Techniques such as dropout and layer normalization are employed to improve model generalization and prevent overfitting. By iteratively adjusting the model's parameters, the training process enables the model to learn underlying patterns and relationships within the data, enhancing its ability to generate coherent and contextually relevant outputs across different modalities.

Evaluation and tuning of large multimodal models are conducted using various metrics tailored to the specific tasks they are designed to perform. For example, BLEU scores are used for text generation tasks, while accuracy is commonly applied for visual recognition tasks to assess performance. Tuning involves adjusting hyperparameters and refining training strategies based on evaluation results to enhance the model's effectiveness. This iterative process ensures that the model can perform a wide range of multimodal tasks with high accuracy and relevance, making it a versatile tool for applications requiring the integration of different types of data.

Large multimodal models represent a significant advancement in machine learning by leveraging sophisticated architectures that combine different neural network types and apply self-attention mechanisms. This enables them to perform complex tasks that require understanding and synthesizing information from diverse data types. Effective preprocessing, rigorous training, and thorough evaluation are crucial to their success, allowing these models to generate coherent and contextually relevant outputs across a wide range of applications.

In accordance with one or more embodiments, other types of models besides LLMs and large multimodal models belong to the broad category of generative models. For example, stochastic models directly incorporate randomness into their structure, making them inherently generative as they can produce a diverse set of outputs for a given input. Generative Adversarial Networks (GANs) learn to generate new data that is indistinguishable from the data they were trained on, using a dual-network architecture that involves a generative component. Variational Autoencoders (VAEs) are explicitly designed for generating new data points by learning a distribution of the input data and encode inputs into a latent space and generate outputs by sampling from this space, making them inherently generative. Sequence-to-sequence models are generative in nature when used with sampling strategies. Although this list of generative model types is not exhaustive, it illustrates the broad use of the term generative model beyond LLMs.

Although generative models can be leveraged for classification tasks, they inherently operate on principles of randomness, leading to a spectrum of possible outcomes in response to identical inputs. Unlike deterministic models that yield a consistent result whenever the same input is given, generative models use the randomness in the data they are trained on to both mimic and diversify from the training data. This diversity makes generative models ideal for generating new and varied data points as well as for tasks that require creativity and novelty.

However, a reliance on randomness creates a trade-off between predictability and flexibility for generative models, potentially making them less predictable in scenarios where uniform outcomes may be expected such as classification tasks.

### 6. Generating Datasets for Training Forecasting Models

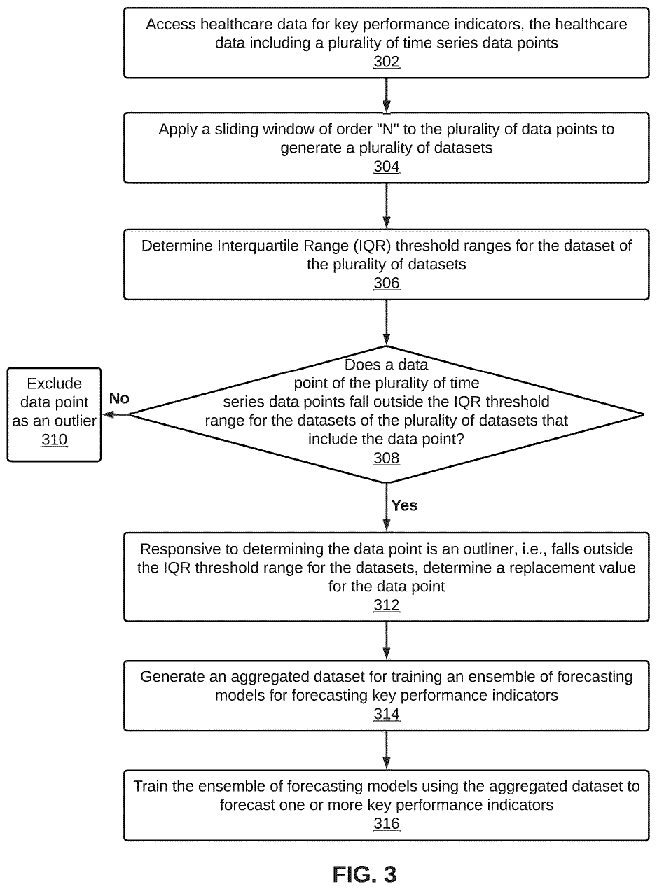

FIG. 3 illustrates an example set of operations for generating datasets for training forecasting models in accordance with one or more embodiments. One or more operations illustrated in FIG. 3 may be modified, rearranged, or omitted all together. Accordingly, the particular sequence of operations illustrated in FIG. 3 should not be construed as limiting the scope of one or more embodiments. The operations illustrated in FIG. 3 may be implemented by machine learning systems and/or processes, such as machine learning engine 142, to optimize model training and performance.

One or more embodiments access healthcare data for key performance indicators, the healthcare data including a plurality of time series data points (Operation 302). Healthcare data for key performance indicators may be retrieved from internal sources, e.g., EHRs, RCM systems, or external sources, e.g., public health databases, third party providers. Many internal and external data sources provide APIs for data access. APIs may be used to extract specific data points, automate reporting, or integrate with analytics. FHIR APIs permit access to standardized healthcare data from EHRs and CMS Blue Button API permits beneficiaries to access Medicare claims data. For structured databases, e.g., data warehouses or ERP systems, SQL queries may be used to extract specific datasets. Healthcare platforms may offer reporting tool that permit users to export data in formats like CSV, Excel, or JSON. Public datasets from organizations like CMS, AHRQ, and NCHS may provide web-based portals for downloading pre-aggregated data. Users may specify datasets, apply filters, and download results directly from the platforms. Some external data sources require a subscription or license.

One or more embodiments prepare the healthcare data for processing. The healthcare data may be prepared as the healthcare data is received. Alternatively, the healthcare data may be prepared at any time during the generation of the dataset. Data preparation includes removing billing entities from the healthcare data with insufficient data points, excluding entities without up-to-date information, and filling dates that are missing KPI values with a KPI value of “0 ” to ensure continuity in the time-series data points.

One or more embodiments apply a sliding window of order “N” to the plurality of data points to generate a plurality of datasets (Operation 304). Using data points from the healthcare data, the system creates overlapping datasets, i.e., windows, of the time series data, each dataset of length “N”, to generate multiple datasets for analysis or model training. The window “slides” over the data, moving one step (or more) at a time, creating multiple overlapping windows of data. The windows are used to generate datasets for forecasting models.

In one or more embodiments, the order “N” refers to the number of consecutive data points included in each window. For example, if “N”=12, then each window will consist of 12 consecutive data points. Starting from the first data point, the system extracts a window of size “N”. The system then moves the window forward by one or more data points and extracts a next window. The system continue this process until reaching the end of the dataset.

In one or more embodiments, the step size of the sliding window may be adjusted to control how much the window moves after each iteration. A step size “1” means the window moves by one data point, resulting in overlapping windows. In this manner, a data point may be included in a maximum of 12 windows or datasets. A step size of “M” means the window moves by “M”data points. When “N”=12″and “M”=12″, resulting in non-overlapping windows.

One or more embodiments determine IQR threshold ranges for the dataset of the plurality of datasets (Operation 306). Initially, the system arranges the data points in each dataset of the “N” datasets in ascending order. The system then determines IQR scores for the datasets. An IQR score for a dataset is equal to Q3 minus Q1, where Q1 is the 25^{th} percentile (lower quartile) and Q3 is the 75^{th} percentile (upper quartile). Q1 may be calculated as a median of the lower half of the dataset and Q3 may be calculated as a median of the upper half of the dataset. Alternatively, Q1 may be the average of the data points extending across the 25^{th} percentile of the dataset and Q3 may be the average of the data points extending across the 75^{th} percentile of the dataset. Q1 may instead be the data point at the 25^{th} percentile of the dataset and Q3 may instead be the data point at the 75^{th} percentile of the dataset.

In one or more embodiments, the IQR scores are then used to calculate the threshold ranges for the datasets. A lower bounds for the threshold ranges is equal to Q1−1.5×IQR score and an upper bounds for the threshold ranges is equal to Q3+1.5×IQR score. Increasing the multiplier, e.g., 2.5, increases the threshold ranges and decreasing the multiplier, e.g., 1, decreases the threshold ranges.

One or more embodiments determine a data point of the plurality of time series data points falls outside the IQR threshold range for the datasets of the plurality of datasets including the data point (Operation 308). The system compares a data point with the threshold ranges for each dataset of the “N” datasets including the data point. The system identifies when data points are within the threshold ranges for the dataset of the “N” datasets including the data point and when the data points are outside the threshold ranges for the datasets of the “N” datasets including the data point.

One or more embodiments, in response to determining a data point is within the IQR threshold range for one or more of the dataset, exclude the data point as an outlier (Operation 310). When the system determines that a data point is within the threshold range for one or more of the datasets of the “N” datasets including the data point, the system identifies the data point as not satisfying the requirements for being an outlier.

One or more embodiments, responsive to determining the data point is an outlier, i.e., falls outside the IQR threshold range for the datasets, determine a replacement value for the data point (Operation 312). When the system determines that a data point is outside the threshold range for every dataset of the “N” datasets including the data point, the system identifies the data point as satisfying the requirements for being an outlier and flags the data point as an outlier.

One or more embodiments, determines a replacement value for the outlier by calculating a median of the neighboring data points of the outlier. The neighboring data points of the outlier may include the “R” neighboring data points before the outlier and the “R” neighboring data points after the outlier. When a neighboring data point is an additional outlier, that neighboring data point is not used in calculating the median. The additional outlier data point may be replaced or may be excluded altogether. The additional outlier data point may be replaced by zero or another suitable integer.

One or more embodiments generate an aggregated dataset for training an ensemble of forecasting models for forecasting key performance indicators (Operation 314). Generating an aggregated dataset includes replacing the outliers with the replacement values, i.e., median of “R” neighbors of the outlier.

One or more embodiments includes associating a features dictionary with the dataset for training the ensemble of forecasting models. The features dictionary may be associated with the dataset using inline documentation, attach as metadata with to data table or frame, or store metadata separately and use a lookup function. Inline documentation, also referred to as code-based association, maintains the features dictionary as a separate object that is referenced whenever metadata about a feature is needed. The features dictionary may be attached as metadata to the data table or frame itself, using custom attributes. Alternatively, the features dictionary may be stored separately, e.g., in a JSON or CSV file, and a helper function may be created to retrieve the metadata.

One or more embodiments using train the ensemble of forecasting models using the aggregated dataset to forecast one or more KPIs (Operation 316). The aggregated training set is applied to various forecasting models. The various forecasting models may employ methodologies including SARIMA, HWES, TBATS, SARIMAX, VARMA, VARMAX, and Prophet.

One or more embodiments combine the forecasts from the various models into a final prediction. The system may determine the final prediction using simple averaging, weighted averaging, or by stacking. Simple averaging takes the average of the forecasts from each model. Weighted averaging assigns weights to each model based on the performance of the model with a validation set. Stacking trains a meta-model, e.g., linear regression, to learn how to best combine the forecasts from the different models.

One or more embodiments incorporate hyperparameter tuning to improve model performance. Techniques like grid search, random search, or Bayesian optimization can be used to explore different configurations of the model and identify the best parameters for optimal performance.

One or more embodiments evaluate the performance of the ensemble forecast. When training multiple models, e.g., using different algorithms or hyperparameters, the system may automatically select the best-performing models for deployment. The system uses various metrics, including MAE, RMSE, and/or MAPE, to evaluate the performance of the forecasting models. The performance of the individual models and the performance of the ensemble of models may be evaluated to determine the best combination of forecasting models.

One or more embodiments use different a different ensemble of models with healthcare data for entities of different characteristics, e.g., size, type of practice, location. A first ensemble of forecasting models may be used with a first entity having a first characteristic and a second ensemble of forecasting models may be used with a second entity having a second characteristic.

One or more embodiments continuously monitor model performance. Model performance may instead be checked when significant changes occur, e.g., market shift or crisis, or at regular intervals.

One or more embodiments validated the trained models against a validation set or via cross-validation or walk forward validation to ensure the models perform well on unseen data. Validation checks for overfitting, underfitting, and generalization ability to ensure the models remain robust in real-world scenarios.

During model updating, one or more embodiments track different versions of the model and ensure that each new version is stored in a version control system. Metrics from the updated model are compared against the old model, and when the new model performs better, the new model can be marked for deployment. When an updated model leads to errors or poor performance in production, the module automatically or manually rolls back to the previous model. Fail-safes ensure that when the new model performs poorly, the system continues to function correctly using the previous version. Each update may be logged and documented, ensuring transparency and accountability in the model updating process. The logs typically include performance metrics of the old and new models, data changes, reasons for retraining, and details of hyperparameter tuning.

### 7. Example IQR Calculations

A detailed example is described below for purposes of clarity. Components and/or operations described below should be understood as one specific example which may not be applicable to certain embodiments. Accordingly, components and/or operations described below should not be construed as limiting the scope of any of the claims.

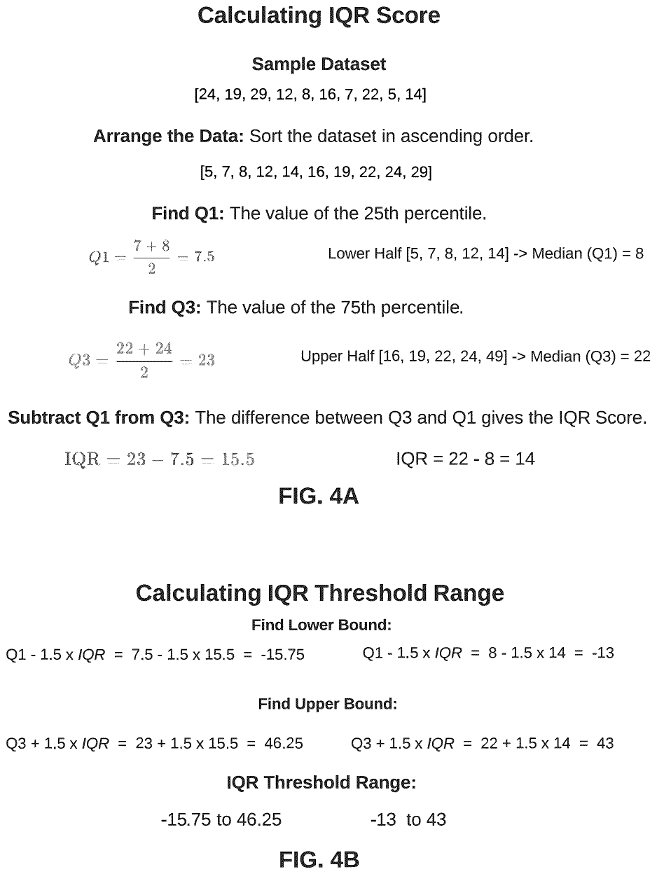

FIG. 4A illustrates how to calculate an IQR score for a sample dataset. The sample dataset includes 10 datapoints 24, 19, 12, 8, 16, 7, 22, 5, and 14. The datapoints of the sample dataset are arranged in ascending order. Datapoint 5 is identified as the first datapoint and datapoint 29 is identified as the last datapoint. The IQR score is equal to Q3−Q1. In the example provided on the left side in FIG. 4A, Q1 is calculated as an average of the data points extending across the 25^{th} percentile of the dataset and Q3 is calculated as an average of the data points extending across the 75^{th} percentile of the dataset. In the example provided on the right side in FIG. 4A, Q1 is calculated as a median of the lower half of the dataset and Q3 is calculated as a median of the upper half of the dataset. Alternatively, Q1 may be the data point at the 25^{th} percentile of the dataset and Q3 may be the data point at the 75^{th} percentile of the dataset. After finding Q1 and Q3 of the dataset, Q1 is subtracted from Q3 to determine the IQR score.

FIG. 4B illustrates how to calculate an IQR threshold range for the sample dataset. The IQR score, in combination with Q1 and Q3 are used to find respective lower and upper bounds for the threshold range. In the example, a multiplier of 1.5 is used to calculate the IQR threshold range, although a larger or smaller multiplier may be used. The lower bound is found by subtracting 1.5×IQR score from Q1 and the upper bound is found by adding 1.5×IQR score to Q3. The IQR threshold range is between the lower bound and the upper bound.

### 8. Example Engineered Features

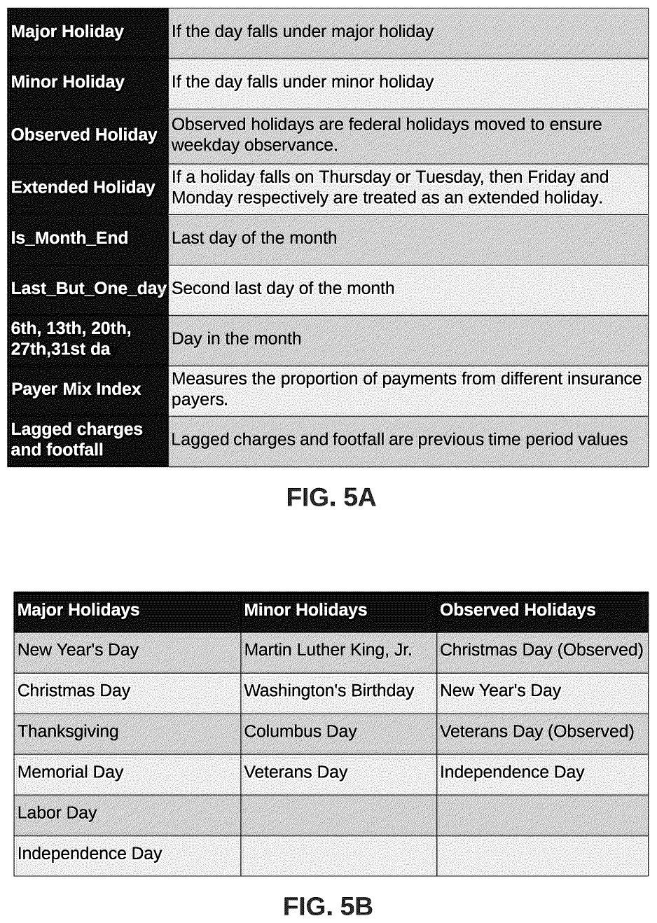

FIGS. 5A and 5B illustrate example engineered features. As shown in FIG. 4A, engineered features include Major Holidays, Minor Holidays, Observed Holidays, Extended Holidays, Month End, Penultimate Day, 6^{th}/13^{th}/20^{th} Day, Payer Mix Index, and Lagged Charges. As shown in FIG. 4B, Major Holidays include New Year's Day, Christmas Day, Thanksgiving, Memorial Day, Labor Day, and Independence Day. Minor Holidays include Martin Luther King, Jr., Washington's Birthday, Columbus Day, and Veterans Day. Observed Holidays include Christmas Day (observed), New Year's Day, Veterans Day (observed), and Independence Day.

### 9. Various Forecasting Models

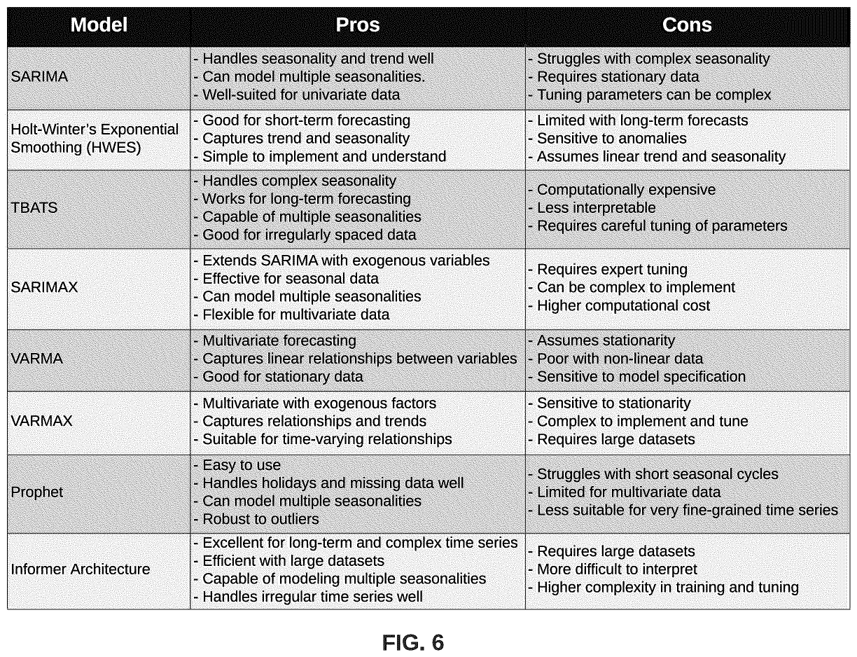

FIG. 6 illustrates various forecasting models for use in the forecasting ensemble. The forecasting models include SARIMA, HWES, TBATS, SARIMAX, VARMA, VARMAX, Prophet, and informer Architecture. Included with the forecasting models are pros and cons for the models.

### 10. Practical Application; Improvements & Advantages

One or more embodiments provide a technical solution to the technical problem of addressing outliers in RCM data that distort ML model outputs. The presence of outliers in RCM data may lead to inaccurate forecasts, inefficient resource allocation, and flawed decision-making. Outliers may cause erroneous revenue cycle predictions, unstable forecasting models, anomalous claim values, and/or data integrity issues. Implementing the outlier detection and handling techniques described herein may improve data quality of the training datasets, leading to a more robust ML model that is able to generate more accurate forecasts despite noise in the input data. Using IQR scores to calculate an IQR threshold range, the system identifies outliers in the RCM data. The system replaces the outliers in RCM data with replacement values. As a result, the training data may more closely represent the real-world data distribution, preventing overfitting and overly biased models.

One or more embodiments provide a technical solution to the technical problem of accounting external factors that affect forecasting and data analysis. External factors, e.g., seasonal trends, lagged charges, payer mix, may impact KPIs and make forecasts unreliable. Not accounting for external factors may cause unpredictable revenue cycle performance, distorted cash flow forecasts, and/or operational inefficiencies. The system accounts for external factors by applying an ensemble of forecasting models. Different forecasting models address different external factors. The system assigns different weights to the various models depending on the importance of the external factors. The system may also account for the external factors by associating a features dictionary with RCM data. Combining multiple models may also help average out the effects of outliers, leading to more accurate AI-driven predictions.

Predictive models for forecasting KPIs for RCM offer significant improvements and advantages for healthcare providers. By enabling proactive management, increasing efficiency, and improving cash flow, predictive analytics enhances the overall revenue cycle. Predictive models for forecasting KPIs can significantly improve the efficiency and accuracy of healthcare revenue cycles. By anticipating changes in RCM metrics, healthcare organizations can proactively manage and optimize their processes, reduce financial losses, and improve cash flow. Predictive models provide actionable insights, enabling data-driven decisions in RCM operations. KPIs forecasted with greater accuracy allow for better alignment of operational strategies with financial goals. Predictive models identify inefficiencies and areas for improvement in the RCM process, reducing manual workload. Automated forecasting reduces reliance on ad hoc reporting and manual analysis, freeing up resources for higher-value tasks.

In one or more embodiments, accurately predicting cash inflows and outflows based on historical billing and payment data allows for optimizing financial planning. Better cash flow management helps maintain liquidity and supports strategic planning for capital expenditures and investments. Timely identification of potential cash shortfalls allows for proactive measures to secure necessary funds, enhancing financial stability.

In one or more embodiments, accurately forecasting workload based on patient volumes, billing cycles, and claims processing times enables organizations to optimize staffing levels. Anticipating workload fluctuations enables more efficient resource allocation, reducing operational bottlenecks. Optimized staffing improves response times, enhances employee satisfaction, and reduces overtime costs. By optimizing staff allocation and improving cash flow, predictive models help reduce overall RCM costs. Proactive measures, informed by predictive analytics, reduce costly reactive interventions and streamline operations. Healthcare providers that leverage predictive models for RCM gain a competitive edge by optimizing their revenue cycle and financial health. Better financial management supports expansion and improved care quality, attracting patients and payers.

In one or more embodiments, accurately predicting patient volume trends and associated revenues enables organizations to support budgeting and resource planning. By aligning resources with expected demand, healthcare providers can improve patient care and reduce wait times. Revenue forecasting allows for more accurate budgeting and helps mitigate the impact of seasonal fluctuations in patient volumes. By understanding how the payer mix will change, organizations can adjust their strategies to maximize reimbursements. Better management of payer mix improves revenue predictability and helps healthcare providers negotiate more favorable terms with payers.

Accurate forecasting of KPIs for RCM supports better cash flow management and enhances revenue stability. Providers can manage expenses, plan for investments, and reduce financial uncertainty by forecasting revenue cycles more effectively. With improved cash flow and resource allocation, providers can invest in quality patient care and reduce wait times.

Predictive modeling supports streamlined billing and payment processes, enhancing patient satisfaction with the financial aspects of care.

One or more embodiments performs AI-driven actions based on the ML model predictions. For example, the system may run a what-if simulation using different hypothetical and/or actual operational parameters as inputs. The what-if simulation may apply the trained ensemble of ML models to the different sets of inputs, outputting a prediction for each different scenario. Based on the ML model outputs, the system may recommend or automate the operational parameters predicted to yield the most optimal KPIs. Example AI-driven insights or actions may include updating or configuring coding software to reduce predicted claim denials, integrating automated eligibility checks and pre-authorization workflows to reduce predicted bottlenecks, modifying a patient registration graphical user interface to streamline patient enrollment, and scheduling automated notifications to reduce predicted payment posting and reconciliation times. Additionally or alternatively, the system provide other AI-driven insights, recommendations, or actions to reduce bottlenecks and/or otherwise optimize workflows.

### 11. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

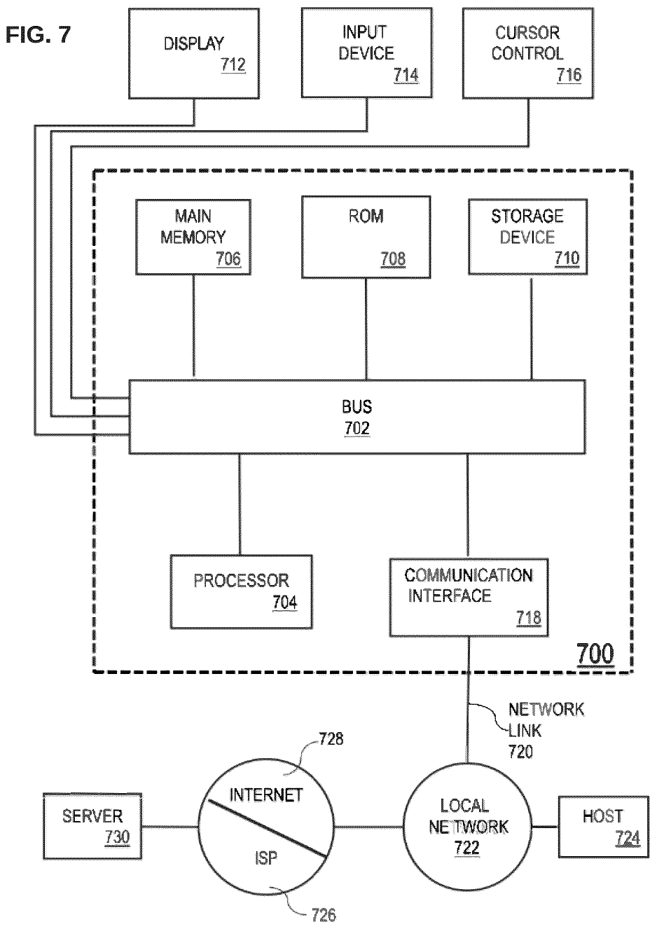

For example, FIG. 7 is a block diagram that illustrates a computer system 700 upon which an embodiment of the disclosure may be implemented. Computer system 700 includes a bus 702 or other communication mechanism for communicating information, and a hardware processor 704 coupled with bus 702 for processing information. Hardware processor 704 may be, for example, a general purpose microprocessor.

Computer system 700 also includes a main memory 706, such as a random access memory (RAM) or other dynamic storage device, coupled to bus 702 for storing information and instructions to be executed by processor 704. Main memory 706 also may be used for storing temporary variables or other intermediate information during execution of instructions to be executed by processor 704. Such instructions, when stored in non-transitory storage media accessible to processor 704, render computer system 700 into a special-purpose machine that is customized to perform the operations specified in the instructions.

Computer system 700 further includes a read only memory (ROM) 708 or other static storage device coupled to bus 702 for storing static information and instructions for processor 704. A storage device 710, such as a magnetic disk, optical disk, or a Solid State Drive (SSD) is provided and coupled to bus 702 for storing information and instructions.

Computer system 700 may be coupled via bus 702 to a display 712, such as a cathode ray tube (CRT), for displaying information to a computer user. An input device 714, including alphanumeric and other keys, is coupled to bus 702 for communicating information and command selections to processor 704. Another type of user input device is cursor control 716, such as a mouse, a trackball, or cursor direction keys for communicating direction information and command selections to processor 704 and for controlling cursor movement on display 712. This input device typically has two degrees of freedom in two axes, a first axis (e.g., x) and a second axis (e.g., y), that allows the device to specify positions in a plane.

Computer system 700 may implement the techniques described herein using customized hard-wired logic, one or more ASICs or FPGAs, firmware and/or program logic which in combination with the computer system causes or programs computer system 700 to be a special-purpose machine. According to one embodiment, the techniques herein are performed by computer system 700 in response to processor 704 executing one or more sequences of one or more instructions contained in main memory 706. Such instructions may be read into main memory 706 from another storage medium, such as storage device 710. Execution of the sequences of instructions contained in main memory 706 causes processor 704 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 710. Volatile media includes dynamic memory, such as main memory 706. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 702. Transmission media can also take the form of acoustic or light waves, such as those generated during radio-wave and infra-red data communications.

Various forms of media may be involved in carrying one or more sequences of one or more instructions to processor 704 for execution. For example, the instructions may initially be carried on a magnetic disk or solid state drive of a remote computer. The remote computer can load the instructions into its dynamic memory and send the instructions over a telephone line using a modem. A modem local to computer system 700 can receive the data on the telephone line and use an infra-red transmitter to convert the data to an infra-red signal. An infra-red detector can receive the data carried in the infra-red signal and appropriate circuitry can place the data on bus 702. Bus 702 carries the data to main memory 706, from which processor 704 retrieves and executes the instructions. The instructions received by main memory 706 may optionally be stored on storage device 710 either before or after execution by processor 704.

Computer system 700 also includes a communication interface 718 coupled to bus 702. Communication interface 718 provides a two-way data communication coupling to a network link 720 that is connected to a local network 722. For example, communication interface 718 may be an integrated services digital network (ISDN) card, cable modem, satellite modem, or a modem to provide a data communication connection to a corresponding type of telephone line. As another example, communication interface 718 may be a local area network (LAN) card to provide a data communication connection to a compatible LAN. Wireless links may also be implemented. In any such implementation, communication interface 718 sends and receives electrical, electromagnetic or optical signals that carry digital data streams representing various types of information.

Network link 720 typically provides data communication through one or more networks to other data devices. For example, network link 720 may provide a connection through local network 722 to a host computer 724 or to data equipment operated by an Internet Service Provider (ISP) 726. ISP 726 in turn provides data communication services through the world wide packet data communication network now commonly referred to as the “Internet” 728. Local network 722 and Internet 728 both use electrical, electromagnetic or optical signals that carry digital data streams. The signals through the various networks and the signals on network link 720 and through communication interface 718, which carry the digital data to and from computer system 700, are example forms of transmission media.

Computer system 700 can send messages and receive data, including program code, through the network(s), network link 720 and communication interface 718. In the Internet example, a server 730 might transmit a requested code for an application program through Internet 728, ISP 726, local network 722 and communication interface 718.

The received code may be executed by processor 704 as it is received, and/or stored in storage device 710, or other non-volatile storage for later execution.

### 12. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected, and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
accessing a dataset of healthcare data for one or more key performance indicators (KPI), the dataset of healthcare data comprising a plurality of time series data points associated with a first KPI;
applying a sliding window of order “N” to the plurality of time series data points to generate a plurality of datasets,
identifying outliers in the plurality of datasets at least by:
determining a first set of “N” datasets of the plurality of datasets that include a first data point,
determining interquartile range (IQR) scores for the datasets of the first set of “N” datasets,
using the IQR scores for the respective datasets of first set of “N” datasets, determining first threshold ranges for the datasets of the first set of “N” datasets,
responsive to the first data point being outside the first threshold ranges for the datasets of the first set of “N” datasets, selecting the first data point as a first outlier of the outliers;
replacing the outliers in the plurality of time series data points with replacement data points to generate an aggregated dataset for the first KPI; and
training at least one machine learning model using the aggregated dataset to forecast the first KPI.

2. The one or more non-transitory computer readable media of claim 1, wherein replacing the outliers in the plurality of time series data points with replacement data points comprises:
identifying a first set of neighboring data points of the first outlier, wherein the first set of neighboring data points comprises data points on a first side of the first outlier and data points on a second side of the first outlier,
determining a first median for the first set of neighboring data points of the first outlier, and
replacing the first outlier with the first median in the aggregated dataset.

3. The one or more non-transitory computer readable media of claim 1, wherein identifying outliers in the plurality of datasets further comprises:
determining a second set of “N” datasets of the plurality of datasets that include a second data point,
determining interquartile range (IQR) scores for the datasets of the second set of “N” datasets,
using the IQR scores for the respective datasets of the second set of “N” datasets, determining second threshold ranges for the datasets of the second set of “N” datasets, and
responsive to the second data point being within the second threshold range for at least one dataset of the second set of “N” datasets, excluding the second data point from selection as an outlier.

4. The one or more non-transitory computer readable media of claim 1, wherein determining the first threshold ranges for the datasets of the first set of “N” datasets comprises:
arranging data points of a first dataset of the first set of “N” datasets in ascending order to generate a first ordered dataset;
determining a Q1 value for the first ordered dataset, wherein the Q1 value is a 25^{th} percentile of the first ordered dataset;
determining a Q3 value of the first ordered dataset, wherein Q3 value is a 75^{th} percentile of the first ordered dataset;
subtracting the Q3 value from the Q1 value to determine an IQR score;
determining a lower threshold of the threshold range by subtracting, 1.5 times the IQR score from the Q1 value; and
determining an upper threshold of the threshold range by adding 1.5 times the IQR score to the Q3 value.

5. The one or more non-transitory computer readable media of claim 1, wherein outliers are excluded from the set of neighboring data points.

6. The one or more non-transitory computer readable media of claim 1, wherein the operations further comprise:
accessing a features dictionary, the features dictionary comprising at least one of:
i. one or more additional KPIs, or
ii. one or more engineered features; and
associating the features dictionary with the aggregated dataset.

7. The one or more non-transitory computer readable media of claim 6, wherein the one or more engineered features comprises two or more of:
i. major holidays,
ii. minor holidays,
iii. extended holiday,
iv. pay mix index,
v. lagged charges, or
vi. lagged footfall.

8. The one or more non-transitory computer readable media of claim 1, wherein the training of at least one machine learning models comprises:
an ensemble of forecasting models, wherein the ensemble of forecasting models comprises:
i. a first plurality of forecasting models trained using the aggregated dataset for forecasting a first KPI value for entities of a first size;
ii. a second plurality of forecasting models trained using the aggregated dataset for forecasting a second KPI value for entities of a second size; and
iii. a third plurality of forecasting models trained using the aggregated dataset for forecasting a third KPI value for entities of a third size,
wherein the first plurality of forecasting models, the second plurality of forecasting models, and the third plurality of forecasting models are different from one another and the first size, the second size, and the third size are different from one another.

9. The one or more non-transitory computer readable media of claim 1, wherein the first KPI comprises one of revenue, cash, or footfall.

10. A method comprising:
accessing a dataset of healthcare data for one or more KPI, the dataset of healthcare data comprising a plurality of time series data points associated with a first KPI;
applying a sliding window of order “N” to the plurality of time series data points to generate a plurality of datasets,
identifying outliers in the plurality of datasets at least by:
determining a first set of “N” datasets of the plurality of datasets that include a first data point,
determining IQR scores for the datasets of the first set of “N” datasets,
using the IQR scores for the respective datasets of first set of “N” datasets, determining first threshold ranges for the datasets of the first set of “N” datasets,
responsive to the first data point being outside the first threshold ranges for the datasets of the first set of “N” datasets, selecting the first data point as a first outlier of the outliers;
replacing the outliers in the plurality of time series data points with replacement data points to generate an aggregated dataset for the first KPI; and
training at least one machine learning model using the aggregated dataset to forecast the first KPI,
wherein the method is performed by at least one device including a hardware processor.

11. The method of claim 10, wherein replacing the outliers in the plurality of time series data points with replacement data points comprises:
identifying a first set of neighboring data points of the first outlier, wherein the first set of neighboring data points comprises data points on a first side of the first outlier and data points on a second side of the first outlier,
determining a first median for the first set of neighboring data points of the first outlier, and
replacing the first outlier with the first median in the aggregated dataset.

12. The method of claim 10, wherein identifying outliers in the plurality of datasets further comprises:
determining a second set of “N” datasets of the plurality of datasets that include a second data point,
determining interquartile range (IQR) scores for the datasets of the second set of “N” datasets,
using the IQR scores for the respective datasets of the second set of “N” datasets, determining second threshold ranges for the datasets of the second set of “N” datasets, and
responsive to the second data point being within the second threshold range for at least one dataset of the second set of “N” datasets, excluding the second data point from selection as an outlier.

13. The method of claim 10, wherein determining the first threshold ranges for the datasets of the first set of “N” datasets comprises:
arranging data points of a first dataset of the first set of “N” datasets in ascending order to generate a first ordered dataset;
determining a Q1 value for the first ordered dataset, wherein the Q1 value is a 25^{th} percentile of the first ordered dataset;
determining a Q3 value of the first ordered dataset, wherein Q3 value is a 75^{th} percentile of the first ordered dataset;
subtracting the Q3 value from the Q1 value to determine an IQR score;
determining a lower threshold of the threshold range by subtracting, 1.5 times the IQR score from the Q1 value; and
determining an upper threshold of the threshold range by adding 1.5 times the IQR score to the Q3 value.

14. The method of claim 10, wherein outliers are excluded from the set of neighboring data points.

15. The method of claim 10, further comprising:
accessing a features dictionary, the features dictionary comprising at least one of:
i. one or more additional KPIs, or
ii. one or more engineered features; and
associating the features dictionary with the aggregated dataset.

16. The method of claim 10, wherein the training of at least one machine learning models comprises:
an ensemble of forecasting models, wherein the ensemble of forecasting models comprises:
i. a first plurality of forecasting models trained using the aggregated dataset for forecasting a first KPI value for entities of a first size;
ii. a second plurality of forecasting models trained using the aggregated dataset for forecasting a second KPI value for entities of a second size; and
iii. a third plurality of forecasting models trained using the aggregated dataset for forecasting a third KPI value for entities of a third size,
wherein the first plurality of forecasting models, the second plurality of forecasting models, and the third plurality of forecasting models are different from one another and the first size, the second size, and the third size are different from one another.

17. The method of claim 10, wherein the first KPI comprises one of revenue, cash, or footfall.

18. A system comprising:
at least one device including a hardware processor;
the system being configured to perform operations comprising:
accessing a dataset of healthcare data for one or more key performance indicators (KPI), the dataset of healthcare data comprising a plurality of time series data points associated with a first KPI;
applying a sliding window of order “N” to the plurality of time series data points to generate a plurality of datasets,
identifying outliers in the plurality of datasets at least by:
determining a first set of “N” datasets of the plurality of datasets that include a first data point,
determining interquartile range (IQR) scores for the datasets of the first set of “N” datasets,
using the IQR scores for the respective datasets of first set of “N” datasets, determining first threshold ranges for the datasets of the first set of “N” datasets,
responsive to the first data point being outside the first threshold ranges for the datasets of the first set of “N” datasets, selecting the first data point as a first outlier of the outliers;
replacing the outliers in the plurality of time series data points with replacement data points to generate an aggregated dataset for the first KPI; and
training at least one machine learning model using the aggregated dataset to forecast the first KPI.

19. The system of claim 18, wherein replacing the outliers in the plurality of time series data points with replacement data points comprises:
identifying a first set of neighboring data points of the first outlier, wherein the first set of neighboring data points comprises data points on a first side of the first outlier and data points on a second side of the first outlier,
determining a first median for the first set of neighboring data points of the first outlier, and
replacing the first outlier with the first median in the aggregated dataset.

20. The system of claim 18, wherein identifying outliers in the plurality of datasets further comprises:
determining a second set of “N” datasets of the plurality of datasets that include a second data point,
determining interquartile range (IQR) scores for the datasets of the second set of “N” datasets,
using the IQR scores for the respective datasets of the second set of “N” datasets, determining second threshold ranges for the datasets of the second set of “N” datasets, and
responsive to the second data point being within the second threshold range for at least one dataset of the second set of “N” datasets, excluding the second data point from selection as an outlier.
